(19) United States
(12) Patent Application Publication
(10) Pub. No.: US 2008/0176923 A1
(43) Pub. Date: Jul. 24, 2008

(54) Proline Derivatives Used as Pharmaceutical Active Ingredients for the Treatment of Tumors

(76) Inventor: Zoser B. Salama, Berlin (DE)

Correspondence Address: JOYCE VON NATZMER;PEQUIGNOT + MYERS LLC, 200 Madison Avenue, Suite 1901, New York, NY, 10016, US

(21) Appl. No.: 10/596,636

(22) PCT Filed: Dec. 18, 2003
(86) PCT No.: PCT/DE03/04211

§ 371 Date: Jun. 19, 2006

(51) Int. Cl. A61K 31/40

(52) U.S. Cl. 514/423; 548/538

(57) ABSTRACT

The invention relates to proline derivatives and their salts, to pharmaceutical agents containing said derivatives and to the use of said agents for treating tumours. The invention also relates to methods for producing said compounds and pharmaceutical agents.

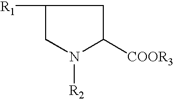

The invention relates to proline derivatives, particularly cis-hydroxyproline derivatives (CHP derivatives) and salts thereof, to pharmaceutical agents comprising the same, and to the use of said agents in the treatment of tumors. Furthermore, the invention relates to the production of the above-mentioned compounds and pharmaceutical agents.

The term “tumor” (or cancer) refers to a complex clinical picture where growth and differentiation of the cells are out of control. As a rule, untreated cancer leads to death. Each year, there are 7 million new incidents of cancer worldwide, with increasing tendency. In the year 2000, the disease was regarded as number 1 cause of death in the industrialized countries.

During the thirties and forties, various amino acids have been tested for their effect on tumors in mice. Among the amino acids used therein were proline and hydroxyproline. Later investigations have shown that the results obtained with mice cannot be applied to human cancerous diseases (DE 35 38 619).

Based on the promising initial tests, efforts have been made time and again in order to provide agents based on proline and hydroxyproline that could be used in cancer prophylaxis and therapy. Thus, for example, the document DE 35 38 619 describes the use of cis-isomers of hydroxyproline in the treatment of carcinomas and related tumors. Various alkyl derivatives of proline and hydroxyproline and their use as drugs in the treatment of cancerous diseases have been disclosed in EP 02 223 850. EP 02 223 850 gives a discussion of various N-methyl derivatives as examples of said alkyl derivatives.

WO 97/33578 describes a drug comprising a combination of cis-hydroxyproline and N-methyl-cis-hydroxyproline for use as therapeutic active substance, especially in cancer therapy. According to WO 97/33578, an anti-tumor effect based on a significant inhibition of cell proliferation has been detected in cell cultures of tumor cells.

The agents disclosed above must be employed at high dosages in order to achieve an effect. Moreover, it was found very difficult to reproduce the results described.

The object of the invention was therefore to provide agents that could be used in an easy, reliable and effective manner in order to inhibit or prevent proliferation, infiltration, invasion, angiogenesis and/or metastasization of cancer cells.

The invention solves the above problem by providing a compound of general formula (I),

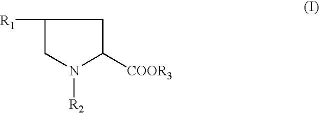

wherein R₁ is a hydroxy, aryl or amino acid group,
R₂ is hydrogen, an alkyl (C₁-C₄), a substituted alkyl (C₁-C₄) group, a dialkyl (C₁-C₄), a cyclohexyl, a phenyl or diphenyl group,
R₃ is an alkyl (C₂-C₅) group,
and/or salts thereof,
with the proviso that, if R₁ is a hydroxy group, R₂ is not a methyl group.

Surprisingly, it was possible to demonstrate that the above-mentioned compounds, i.e., hydroxyproline (CHP) derivatives, can also be employed at high dosages of e.g. more than 0.1 or 0.2 g per kg body weight without substantial side effects. Surprisingly, the new derivatives, especially N-dimethyl esters and phenylaminocarbonyl esters, as well as other claimed compounds, can be used more effectively compared to well-known anti-proliferation agents.

The agents according to the invention can be administered intravenously, e.g. in a range of from 5 to 15 g, and orally in a range of e.g. 50 to 150 g per day. While well-known proline derivatives can be employed particularly for carcinomas, i.e. for tumors of epithelial origin, the agents according to the invention can be used in a variety of diseases substantially determined by cell proliferation or metastasization.

Advantageously, the compounds of the invention can be used particularly as hybrid molecules or in combined agents. For example, the hybrid molecules can be structures comprising the compounds of the invention bound to oxoplatin or to oxoplatin and 5-fluorouracil (5-FU). Using pharmaceutical-technical methods well-known to those skilled in the art, the hybrid molecules can be provided in a way so as to allow their use as prodrug.

The utilization of endocytosis for the cellular uptake of active substances comprising polar compounds is highly effective for some, particularly long-lived, substances, but is very difficult to transfer to more general uses. One alternative is the prodrug concept generally known to those skilled in the art. By definition, a prodrug includes its active substance in the form of a non-active precursor metabolite. It is possible to distinguish between carrier prodrug systems and biotransformation systems. The latter include the active substance in a form requiring chemical or biological metabolization. Such prodrug systems are well-known to those skilled in the art. Carrier prodrug systems include the active substance as such, bound to a masking group which can be cleaved off by a preferably simple controllable mechanism. The inventive function of masking groups in the compounds of the invention is neutralization of the charge for improved reception by cells. When using the compounds of the invention together with a masking group, the latter may also influence other pharmacological parameters, such as oral bioavailability, distribution in tissue, pharmacokinetics, as well as stability to non-specific phosphatases. In addition, delayed release of the active substance may entail a depot effect. Furthermore, modified metabolization may occur, thereby achieving higher efficiency of the active substance or organ specificity. In the event of a prodrug formulation, the masking group, or a linker group binding the masking group to the active substance, is selected in such a way that the prodrug has sufficient hydrophilicity to be dissolved in the blood serum, sufficient chemical and enzymatic stability to reach the site of action, and hydrophilicity suitable for diffusion-controlled membrane transport. Furthermore, it should permit chemical or enzymatic liberation of the active substance within a reasonable period of time and, of course, the liberated auxiliary components should not be toxic. In the meaning of the invention, however, the compound with no mask or no linker and no mask can also be understood as prodrug which initially must be produced via enzymatic and biochemical processes from the incorporated compound in the cell.

In a preferred fashion the amino acids are natural or artificial amino acids such as disclosed in Biochemie; Berg, Tymoczko, Stryer (2003), or other standard textbooks of biology.

In a preferred embodiment of the invention,

R₁ is a hydroxy, phenylamino or an amino acid group,
R₂ is hydrogen, a methyl, dimethyl, cyclohexyl or diphenylmethyl group, and
R₃ is an ethyl, isobutyl group and/or hydrogen.

In a particularly preferred embodiment the phenylamino group of the above compounds comprises modified amino groups, especially phenylaminocarbonyloxy groups. In a particularly preferred fashion the compound is selected from the group comprising 4-hydroxyproline ethyl ester, 4-hydroxy-1,1-dimethylproline ethyl ester iodide, 4-hydroxyproline isobutyl ester, 4-hydroxy-1,1-dimethylproline isobutyl ester iodide, 4-hydroxy-1-cyclohexylproline isobutyl ester, 4-hydroxyl-1-diphenylmethylproline isobutyl ester hydrobromide, 4-hydroxy-1-methylproline, 4-hydroxy-1-methylproline ethyl ester, 4-hydroxy-1-methylproline isobutyl ester, 1-methyl-4-phenylaminocarbonyloxyproline and/or 1-methyl-4-phenylaminocarbonyloxyproline isobutyl ester.

The invention also relates to a pharmaceutical agent comprising a compound according to the invention, optionally together with conventional auxiliaries, preferably pharmaceutically acceptable carriers, adjuvants and/or vehicles.

The compounds of the present invention can be used in the form of salts derived from inorganic or organic acids. For example, such acid salts include the following: acetate, adipate, alginate, aspartate, benzoate, benzenesulfonate, bisulfate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate, and in a particularly preferred fashion the salts of said compounds are iodides, bromides and/or chlorides.

A pharmaceutical agent in the meaning of the invention is any agent in the field of medicine, which can be used in the prophylaxis, diagnosis, therapy, follow-up or aftercare of patients who have come in contact particularly with tumor cells or cancerogens in such a way that a pathogenic modification of the overall condition or of the condition of particular parts of the organism could establish at least temporarily. Thus, for example, the pharmaceutical agent in the meaning of the invention can be a vaccine, an immunotherapeutic or immunoprophylactic agent. The pharmaceutical agent in the meaning of the invention may comprise the compound of the invention or the compound of the invention and/or an acceptable salt or components thereof. For example, salts of inorganic acids can be concerned, such as phosphoric acid, or salts of organic acids. Furthermore, the salts can be free of carboxyl groups and derived from inorganic bases, such as sodium, potassium, ammonium, calcium or iron hydroxides, or from organic bases such as isopropylamine, trimethylamine, 2-ethylaminoethanol, histidine and others. Examples of liquid carriers are sterile aqueous solutions including no additional materials or active ingredients, such as water, or those including a buffer such as sodium phosphate with a physiological pH value or a physiological salt solution or both, e.g. phosphate-buffered sodium chloride solution. Other liquid carriers may comprise more than just one buffer salt, e.g. sodium and potassium chloride, dextrose, propylene glycol, polyethylene glycol or others.

Liquid compositions of said pharmaceutical agents may additionally comprise a liquid phase, also one excluding water. Examples of such additional liquid phases are glycerol, vegetable oils, organic esters or water-oil emulsions. The pharmaceutical composition or pharmaceutical agent typically includes a content of at least 0.1 wt.-% of compounds according to the invention, relative to the overall pharmaceutical composition.

Preferably, 4-hydroxyproline ethyl ester, 4-hydroxy-1,1-dimethylproline ethyl ester iodide, 4-hydroxyproline isobutyl ester, 4-hydroxy-1,1-dimethyl proline isobutyl ester iodide, 4-hydroxy-1-cyclohexylproline isobutyl ester, 4-hydroxy-1-diphenylmethylproline isobutyl ester hydrobromide, 4-hydroxy-1-methylproline, 4-hydroxy-1-methylproline ethyl ester, 4-hydroxy-1-methylproline isobutyl ester, 1-methyl-4-phenylaminocarbonyloxyproline, 1-methyl-4-phenylaminocarbonyloxyproline isobutyl ester, (R)-(+)-α,α-diphenyl-2-pyrrolidinemethanol and/or (S)-(−)-α,α-diphenyl-2-pyrrolidinemethanol are employed in diagnosis, prophylaxis, follow-up, therapy and/or aftercare of diseases associated with cell growth, cell differentiation and/or cell division, especially tumors. The respective dose or dose range for administering the pharmaceutical agent of the invention is in an amount sufficient to achieve the desired prophylactic or therapeutic antiviral effect. The dose should not be selected in such a way that undesirable side effects would dominate. In general, the dose will vary with the age, constitution, sex of a patient, and obviously with respect to the severity of a disease. The individual dose can be adjusted both with respect to the primary disease and with respect to ensuing additional complications. The exact dose can be detected by a person skilled in the art, using well-known means and methods, e.g. by determining the size of the tumor, the number of leukocytes or the like as a function of the dosage or as a function of the vaccination scheme or of the pharmaceutical carriers and the like. Depending on the patient, the dose can be selected individually. For example, a dose of pharmaceutical agent just tolerated by a patient can be one where the local level in plasma or in individual organs ranges from 0.1 to 100,000 μM, preferably between 1 and 1,000 μM. Alternatively, the dose can also be estimated relative to the body weight of the patient. In this event, for example, a typical dose of pharmaceutical agent would be adjusted in a range of more than 0.1 g per kg body weight, preferably between 0.1 and 5,000 g/kg. Furthermore, it is also possible to determine the dose with respect to individual organs rather than the overall patient. For example, this would apply to those cases where the pharmaceutical agent of the invention, incorporated in the respective patient e.g. in a biopolymer, is placed near particular organs by means of surgery. A number of biopolymers capable of liberating the molecules in a desired manner are well-known to those skilled in the art. For example, such a gel may include from 1 to 1000 g of compounds or pharmaceutical agent of the invention per ml gel composition, preferably between 5 and 500 g/ml, and more preferably between 10 and 100 g/ml. In this event, the therapeutic agent will be administered in the form of a solid, gel-like or liquid composition.

In addition to the above-specified concentrations during use of the compounds of the invention, the compounds in a preferred embodiment can be employed in a total amount of 0.05 to 500 g/kg body weight per 24 hours, preferably 5 to 10 g/kg body weight. Advantageously, this is a therapeutic quantity which is used to prevent or improve the symptoms of a disorder or of a responsive, pathologically physiological condition. The amount administered is sufficient to prevent or inhibit growth, metastasization, invasion, infiltration or angiogenesis of the tumor. With respect to their prophylactic or therapeutic potential, the effect of the compounds of the invention on the above tumors is seen e.g. as an inhibition of growth or other. For example, the therapeutic effect can be such that, as a desirable side effect, particular anti-tumor medicaments are improved in their effect or, by reducing the dose, the number of side effects of these medicaments will be reduced as a result of applying the compounds of the invention. Of course, the therapeutic effect also encompasses direct action on the tumor. That is, however, the effect of the compounds of the invention is not restricted to eliminating tumors, but rather comprises the entire spectrum of advantageous effects in prophylaxis and therapy. Obviously, as set forth above, the dose will depend on the age, health and weight of the recipient, degree of the disease, type of required simultaneous treatment, frequency of the treatment and type of the desired effects and side-effects. The daily dose of 0.05 to 500 g/kg body weight can be applied as a single dose or multiple doses in order to furnish the desired results. The dose levels per day can be used in prevention and treatment of a tumor disease. Typically, pharmaceutical agents in particular are used in about 1 to 15 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 5 g/kg body weight being administered in each application. Of course, it is also possible to select a higher content of active substance, e.g. up to a concentration of 500 g/kg. For example, the tablets can also be sustained-release tablets, in which case the number of applications per day is reduced to 1 to 3. The active substance content of sustained-release tablets can be from 3 to 300 g. If the active substance—as set forth above—is administered by injection, the host is preferably contacted 1 to 8 times per day with the compounds of the invention or by using continuous infusion, in which case quantities of from 1 to 400 g per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can easily determine the optimum dosages of active substance required in each case and the type of application of the active substances. In another particularly preferred embodiment of the invention, the compounds of the invention or the pharmaceutical agents are used in a single administration of from 1 to 80, especially from 1 to 30 g/kg body weight. In the same way as the total amount per day, the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in a single application and normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance(s) only in preferred, particular regions of the host.

In another preferred embodiment of the invention the compounds according to the invention can be employed together with at least one other well-known pharmaceutical agent. That is to say, the compounds of the invention can be used in a prophylactic or therapeutic combination in connection with well-known drugs. Such combinations can be administered together, e.g. in an integrated pharmaceutical formulation, or separately, e.g. in the form of a combination of tablets, injection or other medications administered simultaneously or at different times, with the aim of achieving the desired prophylactic or therapeutic effect. These well-known agents can be agents which enhance the effect of the compounds according to the invention. This includes antibacterial or antiviral agents such as benzylpyrimidines, pyrimidines, sulfoamides, rifampicin, tobramycin, fusidinic acid, clindamycin, chloramphenicol and erythromycin. Accordingly, another embodiment of the invention relates to a combination wherein the second agent is least one of the above-mentioned antiviral or antibacterial agents or classes of agents. It should also be noted that the compounds of the invention and combinations can also be used in connection with immune-modulating treatments and therapies.

Typically, there is an optimum ratio of compound(s) of the invention with respect to each other and/or with respect to other therapeutic or effect-enhancing agents (such as trans-port inhibitors, metabolic inhibitors, inhibitors of renal excretion or glucuronidation, such as probenecid, acetaminophen, aspirin, lorazepan, cimetidine, ranitidine, colifibrate, indomethacin, ketoprofen, naproxen etc.) where the active substances are present at an optimum ratio. Optimum ratio is defined as the ratio of compound(s) of the invention to other therapeutic agent(s) where the overall therapeutic effect is greater than the sum of the effects of the individual therapeutic agents. In general, the optimum ratio is found when the agents are present at a ratio of from 10:1 to 1:10, from 20:1 to 1:20, from 100:1 to 1:100 and from 500:1 to 1:500. In some cases, an exceedingly small amount of a therapeutic agent will be sufficient to increase the effect of one or more other agents. In addition, the use of the compounds of the invention in combinations is particularly beneficial to reduce the risk of developing tumor resistance. Of course, the compounds of the invention can be used in combination with other well-known anti-tumor agents. Such agents are well-known to those skilled in the art. Accordingly, the compounds of the invention can be administered together with all conventional agents, especially other drugs, available for use particularly in connection with tumor drugs, either as a single drug or in a combination of drugs. They can be administered alone or in combination with same.

In a preferred fashion the compounds of the invention are administered together with said other well-known pharmaceutical agents at a ratio of about 0.005 to 1. Preferably, the compounds of the invention are administered particularly together with tumor-inhibiting agents at a ratio of from 0.05 to about 0.5 parts and up to about 1 part of said known agents. In this event, antibacterial agents can also be concerned. The pharmaceutical composition can be present in substance or as an aqueous solution together with other materials such as preservatives, buffer substances, agents to adjust the osmolarity of the solution, and so forth. The invention also relates to a kit comprising the compounds of the invention, optionally together with information for combining the contents of the kit. The information for combining the contents of the kit relates to the use of said kit in the prophylaxis and/or therapy of diseases, particularly tumor diseases. For example, the information may also concern a therapeutic regime, i.e., a concrete injection or application schedule, the dose to be administered, or other.

In a preferred fashion the pharmaceutical agent may further include one or more additional agents from the group of antiviral, fungicidal or antibacterial agents and/or immunostimulators or chemotherapeutic agents. Preferably, the antiviral agents are protease inhibitors and/or reverse transcriptase inhibitors. The immunostimulators are preferably bropirimine, anti-human alpha-interferon antibodies, IL-2, GM-CSF, interferons, diethyl dithiocarbamate, tumor necrosis factors, naltrexone, tuscarasol and/or rEPO. The chemotherapeutic agents are preferably alitretinoin, aldesleukin (IL-2), altretamine, all-trans-retinoic acid (tretinoin), aminoglutethimide, anagrelide, anastrozole, asparaginase (E. coli), azathioprine, bicalutamide, bleomycin, busulfan, capecitabine, carboplatin, carmustine, chlorambucil, cisplatin, cladribine (2-CDA), cyclophosphamide, cytarabine, dacarbazine, dactinomycin D, daunorubicin (daunomycin), liposomal daunorubicin, dexamethasone, docetaxel, doxorubicin, liposomal doxorubicin, epirubicin, estramustine phosphate, etoposide (VP-16-213), exemestane, floxuridine, 5-fluorouracil, fludarabine, fluoxymesterone, flutamide, gemcitabine, gemtuzmab, goserelin acetate, hydroxyurea, idarubicin, ifosfamide, imatmib mesylate, irinotecan, α-interferon, letrozole, leuprolide acetate, levamisole-HCl, lomustine, megestrol acetate, melphalan (L-phenylalanine mustard), 6-mercaptopurine, methotrexate, methoxsalen (8-MOP), mitomycin C, mitotane, mitoxantrone, nilutamide, nitrogen mustard (mechlorethamine hydrochloride), α-treotide, paclitaxel, pegaspargase, pentostatin (2′-deoxycoformycin), plicamycin, porfimer, prednisone, procarbazine, rituximab, streptozotocin, tamoxifen, teniposide (VM-26), 6-thioguanine, thalidomide, thiotepa, topotecan, toremifene, trastuzumab, trimetrexate, vinblastine, vincristine and/or vinorelbine. The compounds of the invention can also be used together with immunomodulators or immunostimulators; preferred immunomodulators or immunostimulators are: propirimine, anti-human alpha-interferon antibodies, IL-2, GM-CSF, interferon-α, diethyl dithiocarbamate, tumor necrosis factor, naltrexone, tuscarasol, rEPO and antibiotics such as pentamidinisethionate, but also agents preventing or combating malignant tumors associated with viral diseases. In the method for the treatment of viral, bacterial, fungicidal and/or parasitic infections or of cancer, the compounds of the invention, as set forth above, can be administered together with tolerable carriers, adjuvants or vehicles. Pharmaceutically tolerable carriers, adjuvants and vehicles which can be employed in the drugs of this invention include ion exchangers, aluminum oxide, aluminum stearate, lecithin, self-emulsifying drug delivery systems (SEDDS) such as d-α-tocopherol polyethylene glycol-1000 succinate or other similar polymer delivery matrices, serum proteins such as human serum albumin, buffer substances such as phosphates, glycine, sorbic acids, potassium sorbate, partial glyceride mixtures of saturated vegetable fatty acids, water, salts or electrolytes such as protamin sulfate, disodium hydrogen phosphate, potassium hydrogen phosphate, sodium chloride, zinc salts, colloidal silicon dioxide, magnesium trisilicate, polyvinylpyrrolidone, cellulose-based materials, polyethylene glycol, sodium carboxymethylcellulose, polyacrylates, waxes, polyethylene-polyoxypropylene block polymers, polyethylene glycol and wool fat, but are not restricted thereto. Cyclodextrins such as α-, β- and γ-cyclodextrins or chemically modified derivatives such as hydroxyalkylcyclodextrins, including 2- and 3-hydroxypropyl-β-cyclodextrins or other solubilized derivatives can also be used with advantage to enhance the delivery of the compounds according to the invention. In the context with this method, the compounds of the invention can be administered orally, parenterally, via inhalation spray, topically, rectally, nasally, buccally, vaginally, or by means of an implanted reservoir. Oral administration or administration via injection is preferred as the form of contacting. The drugs of this invention may include any conventional non-toxic, pharmaceutically tolerable carriers, adjuvants or vehicles. In some cases, the pH value of the formulation can be adjusted by means of pharmaceutically tolerable acids, bases or buffers so as to increase the stability of the formulated compound or delivery form thereof. The term “parenteral” as used herein includes subcutaneous, intracutaneous, intravenous, intramuscular, intra-articular, intrasynovial, intrasternal, intrathecal, intralesional and intracranial injection or infusion methods as a form of contacting.

In another preferred embodiment of the invention the carriers are selected from the group comprising fillers, diluents, binders, humectants, disintegrants, dissolution retarders, absorption enhancers, wetting agents, adsorbents and/or lubricants.

The fillers and diluents are preferably starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the wetting agent is preferably cetyl alcohol and glycerol monostearate, the adsorbent is preferably kaolin and bentonite, and the lubricant is preferably talc, calcium and magnesium stearates and solid polyethylene glycols, or mixtures of the materials mentioned above.

In another preferred embodiment of the invention the compounds of the invention are formulated as pharmaceutical agents in the form of a gel, poudrage, powder, tablet, sustained-release tablet, premix, emulsion, brew-up formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can be composed such that release of the active substance(s) takes place only or preferably in a particular area of the intestinal tract, optionally in a delayed fashion, to which end polymer substances and waxes can be used as embedding materials.

Preferably, the compounds or drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate are added. For oral administration in the form of capsules, diluents that can be used include lactose and dried corn starch. In oral administration of aqueous suspensions the active substance is combined with emulsifiers and suspending agents. Also, specific sweeteners and/or flavors and/or coloring agents can be added, if desired.

The active substance(s) can also be present in microencapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substance(s), solutions and emulsions may include conventional carriers such as solvents, solubilizers, and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substance(s), suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar and tragacanth or mixtures of these substances.

The drugs can be present in the form of a sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the active substances of formula (I) should be present in the above-mentioned pharmaceutical preparations at a concentration of about 0.1 to 99.5, more preferably about 0.5 to 95 wt.-% of the overall mixture.

In addition to the compounds of formula (I) and (II), the above-mentioned pharmaceutical preparations may include further pharmaceutical active substances. The production of the pharmaceutical preparations specified above proceeds in a usual manner according to well-known methods, e.g. by mixing the active substance(s) with the carrier material(s).

The above-mentioned preparations can be applied in humans and animals on an oral, rectal, parenteral (intravenous, intramuscular, subcutaneous), intracisternal, intravaginal, intraperitoneal route, locally (powders, ointment, drops), and used in therapy. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Furthermore, gels, powders, tablets, sustained-release tablets, premixes, concentrates, granulates, pellets, boli, capsules, aerosols, sprays, inhalants can be used in humans and animals. Moreover, the compounds of the invention can be incorporated in other carrier materials such as plastics (plastic chains for local therapy), collagen or bone cement.

In another preferred embodiment of the invention the compounds of the invention are incorporated in a preparation at a concentration of 0.1 to 99.5, preferably 0.5 to 95, and more preferably 20 to 80 wt.-%. That is, the compounds of the invention are present in the above-specified pharmaceutical formulations, e.g. tablets, pills, granulates and others, at a concentration of preferably 0.1 to 99.5 wt.-% of the overall mixture. The amount of active substance, i.e., the amount of an inventive compound combined with the carrier materials to produce a single dosage form, can vary depending on the host to be treated and on the particular type of administration. Once the condition of a host or patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a viral disease is combated as effectively as possible or the onset of such a disease is prevented by a prophylactic administration. Concentration and type of application can be determined by a person skilled in the art using routine tests. Preferred applications of the compounds of the invention are oral application in the form of powders, tablets, fluid mixtures, drops, capsules or the like, rectal application in the form of suppositories, solutions and the like, parenteral application in the form of injections, infusions and solutions, inhalation of vapors, aerosols and powders and pads, and local application in the form of ointments, pads, dressings, lavages and the like. Contacting with the compounds according to the invention is preferably effected in a prophylactic or therapeutic fashion. In prophylactic administration, development of tumors is to be prevented. In therapeutic contacting, a tumor disease is already existing, and the cancer cells already present in the body should either be destroyed or inhibited in their growth. Other forms of application preferred for this purpose are e.g. subcutaneous, sublingual, intravenous, intramuscular, intraperitoneal and/or topical ones.

For example, the suitability of the selected form of application, of the dose, application regimen, selection of adjuvant and the like can be determined by taking serum aliquots from the patient or by using imaging methods in the course of the treatment procedure. Alternatively or concomitantly, the condition of the liver, but also, the amount of T cells or other cells of the immune system can be determined in a conventional manner so as to obtain a general survey on the immunologic constitution of the patient and, in particular, the constitution of organs important to the metabolism, particularly of the liver. Additionally, the clinical condition of the patient can be observed for the desired effect, especially the anti-tumor effect. Tumor diseases can be associated with further infections, e.g. bacterial or mycotic, for which reason additional clinical co-monitoring of the course of such concomitant infections is also possible. Where insufficient anti-tumor effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration. In addition to oral ingestion, e.g. intramuscular or subcutaneous injections or injections into the blood vessels can be envisaged as other preferred routes of therapeutic administration of the compounds according to the invention. At the same time, supply via catheters or surgical tubes can also be used.

Accordingly, the invention also relates to the use of the compounds in diagnosis, prophylaxis, follow-up, therapy, and/or aftercare of diseases associated with cell growth, cell differentiation and/or cell division.

In a preferred embodiment the disease associated with cell growth, cell differentiation and/or cell division is a tumor. In a particularly preferred fashion the tumor is a solid tumor or a leukemia.

In a preferred embodiment the cancerous disease or tumor being treated or prophylactically prevented, or whose recurrence is prevented, is selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchioalveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarian carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prophylactically prevented, or whose recurrence is prevented, is selected from the following group of cancerous diseases or tumor diseases: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prophylactically prevented, or whose reappearance is prevented, is selected from the group comprising cancerous diseases or tumor diseases such as mammary carcinomas, gastrointestinal tumors, including colon carcinomas, stomach carcinomas, large intestine cancer and small intestine cancer, pancreas carcinomas, ovarian carcinomas, liver carcinomas, lung cancer, renal cell carcinomas, multiple myelomas.

In a specific embodiment of the invention the compounds or the pharmaceutical composition is used in a combined therapy, especially in the treatment of tumors. In a particularly preferred fashion, said combined therapy comprises a chemotherapy, treatment with cytostatic agents and/or a radiotherapy.

In a particularly preferred embodiment of the invention the combined therapy is an adjuvant, biologically specified form of therapy. Even more preferably, said form of therapy is an immune therapy. In a likewise particularly preferred fashion, said combined therapy is a gene therapy.

In the meaning of the invention, gene therapy is a form of treatment using natural or recombinantly engineered nucleic acid constructs, single gene sequences or complete gene or chromosome sections or encoded transcript regions, derivatives/modifications thereof, with the objective of a biologically based and selective inhibition or reversion of disease symptoms and/or of the causal origin thereof, in special cases this being understood to involve inhibition of a target molecule on a nucleic acid level, especially transcript level, which has been overexpressed in the course of a disease.

Various combination therapies, especially for the treatment of tumors, are well-known to those skilled in the art. For example, a treatment with cytostatic agents or e.g. irradiation of a particular tumor area can be envisaged within the scope of a combination therapy, and this treatment is combined with a gene therapy, using the compound of the invention as an anti-cancer agent. However, the agents according to the invention can also be used in combination with other anti-cancer agents. Accordingly, in a particularly preferred fashion the compound can be used to increase the sensitivity of tumor cells to cytostatic agents and/or radiation. Furthermore, a preferred use of the compound is in inhibiting the viability and the proliferation rate of cells and/or inducing apoptosis and cell cycle arrest.

The invention also relates to a method for the production of the compounds according to the invention. Thus, for example, 1-methyl-4-phenylaminocarbonyloxyproline ethyl ester is obtained by reacting 4-hydroxy-1-methylproline ethyl ester and phenyl isocyanate in acetonitrile.

The inventive compound 1-methyl-4-phenylaminocarbonyloxyproline isobutyl ester is obtained by reacting 4-hydroxy-1-methylproline isobutyl ester and phenyl isocyanate in acetonitrile.

4-Hydroxy-1-methylproline is obtained by reacting 4-hydroxyproline in formalin with Pd/C in a hydrogenation apparatus.

4-Hydroxy-1-methylproline ethyl ester is obtained by reacting 4-hydroxyproline ethyl ester and formalin in ethanol.

4-Hydroxy-1-methylproline isobutyl ester is obtained by reacting formalin, Pd/C and ethanol and 4-hydroxyproline isobutyl ester.

4-Hydroxy-1-methylproline isobutyl ester is obtained by reacting formalin and 4-hydroxyproline isobutyl ester in the presence of Pd/C in ethanol.

The derivatives of 4-hydroxyproline are obtained as follows. cis-4-Hydroxy-L-proline ethyl ester is obtained by contacting 4-hydroxyproline with HCl in ethanol (see example).

cis-4-Hydroxy-L-proline isobutyl ester is obtained by reacting 4-hydroxyproline in isobutanol, the purification being effected in analogy to 4-hydroxyproline ethyl ester.

4-Hydroxy-1,1-dimethylproline ethyl ester iodide is obtained by dissolving hydroxyproline ethyl ester in acetonitrile and adding methyl iodide and triethylamine.

4-Hydroxy-1,1-dimethylproline isobutyl ester iodide is obtained by reacting 4-hydroxyproline isobutyl ester and methyl iodide in triethylamine and acetonitrile.

4-Hydroxy-1-alkylproline ester bromide is obtained by suspending 4-hydroxyproline ester in acetonitrile and contacting with the corresponding alkyl bromide.

4-Hydroxy-1-cyclohexylproline isobutyl ester is formed by dissolving the hydrobromide in chloroform and subsequent drying in ammonia gas.

4-Hydroxy-1-diphenylmethyl proline isobutyl ester hydrobromide is obtained in analogy to 4-hydroxy-1,1-dimethylproline isobutyl ester iodide.

The invention also relates to the use of the compounds to inhibit collagen IV and/or glutathione S transferase (GST), said compounds being those described above for cancer therapy.

GST inhibition or lowering and/or collagen IV inhibition or lowering in a cell culture or in an organism has a number of consequences. In organisms or in vitro cultures, for example, GST is capable of binding GSH so as to prepare the latter for extracellular transport. In the event of a tumor cell, this would imply the following: GST binds oncogens or other components of the tumor cell to GSH, conveying them into the extracellular region, which—among other things—gives rise to the spreading effect and, as a consequence, formation of metastases. As a result of increased GSH binding, the latter is no longer available for other cellular processes, and this gives rise to pathological changes in the cell. In addition, binding of tumor cell fragments results in a different way of information processing within the cell, so that functions proceed in a different way, thereby initiating or promoting transformation of the cell. Moreover, the processes mentioned above promote apoptosis.

However, higher tolerance to carcinogens and inhibition of carcinogenesis are not the only consequences of inhibition effected by CHP derivatives. Other secondary responses of such inhibition comprise e.g. therapy or alleviation of autoimmune diseases, regeneration of cells following chemotherapy or in parallel with chemotherapy, alleviation of the ageing process by removing interfering radicals, treatment of infectious diseases as well as metabolic diseases, especially of the liver, pancreas, intestine and/or stomach.

In a preferred fashion, such secondary processes of GST inhibition are associated with other chemical secondary processes of collagen IV inhibition. In particular, the secondary processes of collagen IV inhibition result from the fact that tumor cells dock via the main collagen domain of this glycoprotein, thus infiltrating and penetrating the cells. However, collagen inhibition not only results in diminished metastasizing and infiltration and invasion in tumor diseases, but also exhibits therapeutic effects in all inflammatory diseases wherein normal tissue is reconstructed into connective tissue, e.g. in lung fibrosis, liver cirrhosis, pancreatic fibrosis and/or glomerulosclerosis. Furthermore, collagen IV inhibition shows a positive influence on scleroderma/Marfan syndrome, vascular diseases, metabolic diseases, autoimmune diseases, and neurological diseases wherein nervous tissue is turned into connective tissue, so-called glioses, as is the case in Alzheimer's disease, for example. In addition to inhibiting collagen IV by CHP, it is obviously possible—particularly in the last-mentioned diseases—to administer parallel medications inhibiting fibrosis, e.g. bleomycin/busulfan, in the form of a supportive/additive therapy.

The invention also relates to a method of inhibiting collagen IV and/or GST in an organism and/or in a sample, in which method the organism or a sample is contacted with CHP. For example, the method can be used in a combination therapy, by means of which cells in an organism regenerate following chemotherapy. For example, contacting of CHP with the organism or the sample to be treated can be effected orally, subcutaneously, intravenously, intramuscularly, intraperitoneally, vaginally, rectally, topically and/or sublingually.

The invention also relates to an anti-collagen IV agent and/or anti-GST agent or collagen IV- or GST-lowering agent comprising CHP, optionally together with standard auxiliary agents. More specifically, these standard auxiliary agents are pharmaceutically acceptable carriers, adjuvants and/or vehicles, said carriers being selected from the group comprising fillers, diluents, binders, humectants, disintegrants, dissolution retarders, absorption enhancers, wetting agents, adsorbents and/or lubricants. The collagen IV-lowering agent or inhibitor or the GST-lowering agent or inhibitor comprising CHP derivatives can be prepared and/or used in the form of a gel, poudrage, powder, tablet, sustained-release tablet, premix, emulsion, brew-up formulation, drops, concentrate, infusion solutions, granulate, syrup, pellet, bolus, capsule, aerosol, spray and/or inhalant. In a preferred fashion, CHP is present in a formulation at a concentration of from 0.1 to 99.5, preferably from 0.5 to 95, and more preferably from 1 to 80 wt.-%. In a particularly preferred fashion the formulation is an infusion solution wherein CHP is present in a range of from 1 to 2 wt.-%.

In another embodiment of the invention, CHP derivatives are employed in overall amounts of from 0.05 to 1000 mg per kg body weight, preferably from 5 to 450 mg per kg body weight per 24 hours.

The collagen IV inhibitor or GST inhibitor or CHP derivatives alone can be used in such a way that 0.1 to 100 g is administered per day and patient. Of course, splitting the daily dose and contacting the correspondingly split amount 2, 4, 6 or 10 times or more with the organism can also be envisaged.

Inhibition of collagen IV and/or GST, preferably (GST, by CHP derivatives is preferably used in the treatment of (i) inflammations, especially preferably (ii) autoimmune diseases.

(i) Inflammations in the meaning of the invention are reactions of the organism, mediated by the connective tissue and blood vessels, to an external or internally triggered inflammatory stimulus, with the purpose of eliminating or inactivating the latter and repairing the tissue lesion caused by said stimulus. A triggering effect is caused by mechanical stimuli (foreign bodies, pressure, injury) and other physical factors (ionizing radiation, UV light, heat, cold), chemical substances (alkaline solutions, acids, heavy metals, bacterial toxins, allergens, and immune complexes), and pathogens (microorganisms, worms, insects), or pathologic metabolites, derailed enzymes, malignant tumors. The process begins with a brief arteriolar constriction (as a result of adrenaline effect), with inadequate circulation and tissue alteration, followed by development of classical local inflammatory signs (cardinal symptoms, according to GALEN and CELSUS), i.e., from reddening (=rubor; vascular dilation caused by histamine), heat (=calor; as a result of local increase of metabolism), swelling (=turgor; as a result of secretion of protein-rich liquor from vessel walls changed by histamine, among other things, supported by decelerated blood circulation in the sense of a prestasis up to stasis), pain (=dolor; as a result of increased tissue tension and algogenic inflammation products, e.g. bradykinin), and functional disorders (=functio laesa). The process is accompanied by disorders in the electrolyte metabolism (transmineralization), invasion of neutrophilic granulocytes and monocytes through the vessel walls (cf., leukotaxis), with the purpose of eliminating the inflammatory stimulus and the damaged to necrotic cells (phagocytosis); furthermore, invasion of lymphocyte effector cells, giving rise to formation of specific antibodies against the inflammatory stimulus (immune reaction), and of eosinophiles (during the phase of healing or—at a very early stage—in allergic-hyperergic processes). As a result of the activation of the complement system occurring during the reaction, fragments (C3a and C5a) of this system are liberated which—like histamine and bradykinin—act as inflammation mediators, namely, in the sense of stimulating the chemotaxis of the above-mentioned blood cells; furthermore, the blood coagulation is activated. As a consequence, damage (dystrophia and coagulation necrosis) of the associated organ parenchyma occurs. Depending on the intensity and type of the inflammation, the overall organism responds with fever, stress (cf., adaptation syndrome), leukocytosis and changes in the composition of the plasma proteins (acute-phase reaction), giving rise to an accelerated erythrocyte sedimentation. Preferred inflammations in the meaning of the invention are suppurative, exudative, fibrinous, gangrenescent, granulomatous, hemorrhagic, catarrhal, necrotizing, proliferative or productive, pseudomembranous, serous, specific and/or ulcerous inflammations.

(ii) Autoimmune diseases in the meaning of the invention are diseases entirely or partially due to the formation of autoantibodies and their damaging effect on the overall organism or organ systems, i.e., due to autoaggression. A classification into organ-specific, intermediary and/or systemic autoimmune diseases can be made. Preferred organ-specific autoimmune disease are HASHIMOTO thyroiditis, primary myxedema, thyrotoxicosis (BASEDOW disease), pernicious anemia, ADDISON disease, myasthenia gravis and/or juvenile diabetes mellitus. Preferred intermediary autoimmune diseases are GOODPASTURE syndrome, autoimmune hemolytic anemia, autoimmune leukopenia, idiopathic thrombocytopenia, pemphigus vulgaris, sympathetic ophthalmia, primary bile cirrhosis, autoimmune hepatitis, ulcerative colitis and/or SJOGREN syndrome. Preferred systemic autoimmune diseases are rheumatoid arthritis, rheumatic fever, systemic lupus erythematosus, dermatomyositis/polymyositis, progressive systemic sclerosis, WEGENER granulomatosis, panarteritis nodosa and/or hypersensitivity angiitis. Typical autoimmune diseases are thyrotoxicosis, thyroid-caused myxedema, HASHIMOTO thyroiditis, generalized endocrinopathy, pernicious anemia, chronic gastritis type A, diseases of single or all corpuscular elements of the blood (for example, autoimmune hemolytic anemia, idiopathic thrombocytopenia or thrombocytopathy; idiopathic leukopenia or agranulocytosis), pemphigus vulgaris and pemphigoid, sympathetic ophthalmia, and numerous forms of uveitis, primarily biliary liver cirrhosis and chronic aggressive autoimmune hepatitis, diabetes mellitus type I, CROHN disease and ulcerative colitis, SJOGREN syndrome, ADDISON disease, lupus erythematosus disseminatus and discoid form of said disease, as dermatomyositis and scleroderma, rheumatoid arthritis (=primarily chronic polyarthritis), antiglomerular basement membrane nephritis. The basis is an aggressive immune reaction due to breakdown of the immune tolerance to self-determinants and a reduction of the activity of T suppressor cells (with lymphocyte marker T8) or an excess of T helper cells (with lymphocyte marker T4) over the suppressor cells; furthermore, formation of autoantigens is possible e.g. by coupling of host proteins to haptens (e.g. drugs), by ontogenetic tissue not developing until self-tolerance has developed, by protein components demasked as a result of conformational changes of proteins in connection with e.g. infection by viruses or bacteria; and by new proteins formed in association with neoplasias. Also preferred is the treatment of all the above-mentioned cancerous diseases via inhibition of collagen IV and/or GST.

Without intending to be limiting, the invention will be explained in more detail with reference to the following examples.

### 1. Hydroxyproline Derivatives

### Preparation of 1-methyl-4-phenylaminocarbonyloxyproline ethyl ester (A-1-23)

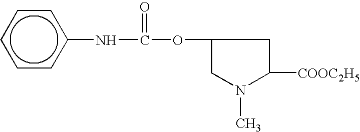

### Batch:

430 mg (0.0025 mol) of 4-hydroxy-1-methylproline ethyl ester, 300 mg of phenyl isocyanate, 30 ml of acetonitrile.

### Synthesis:

The starting materials are dissolved in acetonitrile and refluxed for about 5 hours. Following cooling to room temperature, the solvent is removed in vacuum, the raw product is dissolved in acetone and precipitated with ether/heptane.

Yield: 200 mg (27% of theoretical amount)

m.p.: 178-80° C.

### Preparation of 1-methyl-4-phenylaminocarbonyloxyproline isobutyl ester (A-2-23)

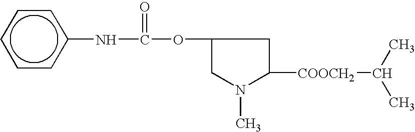

### Batch:

500 mg (0.0025 mol) of 4-hydroxy-1-methylproline isobutyl ester, 300 mg of phenyl isocyanate, 30 ml of acetonitrile.

### Synthesis:

In analogy to A-1-23

### Preparation of 4-hydroxy-1-methylproline (A-0-21)

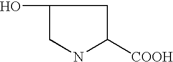

### Synthesis:

4 g of 4-hydroxyproline, 4 ml of formalin, 200 mg of Pd/C and 250 ml of ethanol are agitated in a hydrogenation apparatus under hydrogen atmosphere (normal pressure, room temperature) for about 36 hours (reductive amination). Thereafter, the catalyst is filtrated off, the filtrate is concentrated to near dryness, and the reaction product is precipitated by addition of about 250 ml of acetone (this purification procedure is repeated twice, if necessary); the product is sucked off and dried in vacuum.

Yield: 4.0 g (about 91% of theoretical amount)

m.p.: 190° C.

### Preparation of 4-hydroxy-1-methylproline ethyl ester (A-1-21)

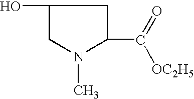

### Batch:

2 g of 4-hydroxyproline ethyl ester, 2 g of formalin, 200 mg of Pd/C, 150 ml of ethanol.

### Synthesis:

In analogy to A-0-21

Yield: 1.4 g (about 64% of theoretical amount)

m.p.: 204° C.

### Preparation of 4-hydroxy-1-methylproline isobutyl ester (A-2-21)

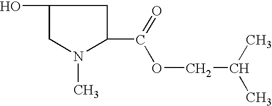

### Batch:

2 g of 4-hydroxyproline isobutyl ester, 2 g of formalin, 200 mg of Pd/C, 150 ml of ethanol.

### Synthesis:

In analogy to A-0-21.

Yield: 1.5 g (about 65% of theoretical amount)

m.p.: 220° C.

Derivatives of 4-hydroxyproline

### Preparation of cis-hydroxy-L-proline ethyl ester (A-1)

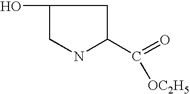

### Synthesis:

Dry HCl gas is introduced into a suspension of 20 g (0.15 mol) of 4-hydroxyproline in 400 ml of anhydrous ethanol with stirring and ice cooling (about 2 hours) until 4-hydroxyproline is dissolved, and additional HCl gas is introduced once a day (about 5 to 10 minutes).

Work-up/purification: After removal of the alcohol in vacuum, the remaining ester hydrochloride is dissolved in chloroform/methanol (8:2), dry NH₃ gas is introduced (about 5 min), the solvent is removed in vacuum, and the product mixture (proline ester+NH₄Cl) is treated with warm chloroform. After sucking off the NH₄Cl, the filtrate is concentrated to dryness in vacuum.

Yield: 18 g (75.5% of theoretical amount)

m.p.: 115° C.

### Preparation of cis-hydroxy-L-proline isobutyl ester (A-2)

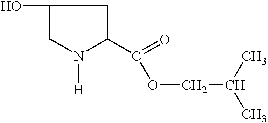

### Batch:

10 g (0.075 mol) of 4-hydroxyproline, 250 ml of isobutanol (dry)

### Synthesis:

Work-up/purification: in analogy to 4-hydroxyproline ethyl ester.

Yield: 11 g (78.6% of theoretical amount)

m.p.: 139° C.

### Preparation of 4-hydroxy-1,1-dimethylproline ethyl ester iodide (A-1-01)

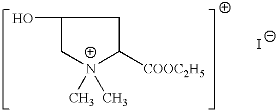

### Synthesis:

Hydroxyproline ethyl ester (0.8 g) is dissolved in 30 ml of acetonitrile and added with 0.6 g of methyl iodide and 1 ml of triethylamine. After standing overnight (room temperature), the reaction mixture is briefly heated (the reaction product completely dissolving in the acetonitrile) and immediately filtrated while hot (removal of triethylammonium iodide). The acetonitrile is removed in vacuum, and the remaining solid-crystalline final product is dried in vacuum.

Yield: 600 g (44.4% of theoretical amount)

m.p.: 118-120° C.

### Preparation of 4-hydroxy-1,1-dimethylproline isobutyl ester iodide (A-2-01)

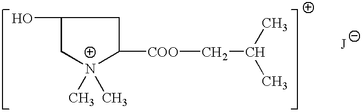

### Batch:

0.9 g of 4-hydroxyproline isobutyl ester (A-2), 1 g of methyl iodide, 1 ml of triethylamine, 40 ml of acetonitrile.

### Synthesis:

In analogy to A-1-01.

Yield: 0.6 g (36.6% of theoretical amount)

m.p.: 180° C.

### Preparation of 4-hydroxy-1-alkylproline ester bromide

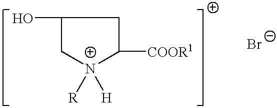

### General Protocol:

The respective 4-hydroxyproline ester (0.01 mol) is suspended in 40 ml of acetonitrile and, following addition of 0.01 mol of the corresponding alkyl bromide, refluxed for 5 hours. After cooling to room temperature, the reaction mixture is added to 400 ml of ether and cooled overnight (about −20° C.). This is sucked off and dried in vacuum.

### Preparation of 4-hydroxy-1-cyclohexylproline isobutyl ester (A-2-03)

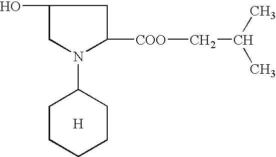

### Synthesis:

The corresponding hydrobromide (1.7 g) is dissolved in 150 ml of warm chloroform, followed by introduction of dry ammonia gas for about 3 minutes. After cooling to room temperature, the precipitated ammonium bromide is sucked off, the chloroform is removed in vacuum, and the remaining raw product is eventually recrystallized from heptane.

Yield: 0.8 g (61.1% of theoretical amount)

no m.p. (pasty)

### Preparation of 4-hydroxy-1-diphenylmethylproline isobutyl ester hydrobromide (A-2-04)

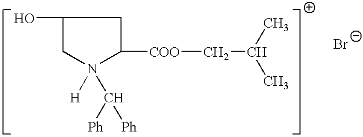

### Synthesis:

See A-2-01

Yield: 2.8 g (64.5% of theoretical amount)

m.p.: 49-147° C.

### 2. Effects of the Synthesized Hydroxyproline Derivatives on Tumor Cell Proliferation

The compounds of the invention were tested using the pancreas tumor cell lines MIYPaCa2 and BxPC3, the breast cancer cell lines MDA-MB-435 and BT20, as well as the colon cancer cell lines Colo205 and HT29. The cells were placed in culture medium (RPMI-1640 with 10% fetal calf serum and 4 mM glutamine) in 96-well microtiter plates to make 10,000 cells per well. The inventive components to be tested were diluted in microtiter plates according to the well-known procedure and incubated for 4 days under cell culture conditions (37° C., 5% CO₂).

Following incubation, the proliferation was tested using a tetrazolium-based EZ4U kit from Biomedica (Vienna, Austria). The optical density (OD) of each well was determined by means of an ELISA Reader, and the control medium obtained was set to 100% (OD_{490 nm}=0.5 to 1.5). The values in Table 1 are given in %, showing the inhibition of cellular proliferation, the concentration being 400 μg/ml (higher value) and 200 μg/ml (lower value).

TABLE 1

| | A0.21 | A1.21 | A1.23 | A2.21 | A2.23 | CHP |
|---|---|---|---|---|---|---|
| MIA- | 22.8 | 38.8 | 8.3 | 23.9 | 0.3 | 51.3 |
| PaCa2 | 21.1 | 7.4 | 16.0 | 4.5 | 14.2 | 34.5 |
| Colo205 | 16.3 | 32.7 | 7.3 | 10.0 | 14.1 | 78.2 |
| | 21.1 | 23.7 | 17.2 | 14.1 | 19.2 | 60.0 |
| BxPC3 | 16.2 | 13.4 | 0 | 4.9 | 2.1 | 44.5 |
| | 21.9 | 25.0 | 17.8 | 18.2 | 15.4 | 27.8 |
| MDA- | 23.2 | 53.5 | 4.5 | 3.1 | 8.6 | 39.2 |
| MB435 | 26.9 | 15.6 | 8.7 | 4.5 | 5.5 | 19.9 |
| BT20 | 14.9 | 80.1 | 1.3 | 3.9 | 0 | 10.6 |
| | 18.3 | 22.2 | 4.4 | 5.2 | 0 | 6.5 |
| HT29 | 4.7 | 0.5 | 1.9 | 1.3 | −5.3 | 15.7 |
| | 4.8 | 7.3 | 8.5 | 4.1 | −5.0 | 1.7 |

CHP has the highest activity (40±10.1% inhibition; mean value±SEM for all 6 cell lines at 400 μg/ml, followed by A1.21 (36.5±11.4) and A0.21 (16.3±2.7) and A1.23, A2.21, A2.23 with activities below 8%. A1.21 has a spectrum which is different from that of CHP and has a much lower activity at the lower concentration compared to CHP.

Surprisingly, cis-hydroxy-N-methylproline ethyl ester showed a higher activity for particular cell lines such as MDA-MB435 and BT20, both being breast cancer cell lines. In further tests, substances were dissolved in water, and their effect on the colon adenocarcinoma cell line Colo205 and pancreas adenocarcinoma cell line BxPC3 as targets was tested. The results obtained, in IC₅₀ concentrations in μg/ml, are illustrated in Table 2.

TABLE 2

| Substance | IC₅₀ Colo205 | IC₅₀ BxPC3 |
|---|---|---|
| A-1 | 50 | 5 |
| A1-01 | >>400 | 70 |
| A2 | 18 | 15 |
| A2-01 | >400 | 50 |
| A2-03 | 50 | 50 |
| A2-04 | 6.2 | 12 |
| CHP | 90 | 400 |

Surprisingly, it was found that the effect of cis-4-hydroxy-L-proline ethyl ester (A1), cis-4-hydroxy-L-proline isobutyl ester (A2), cis-4-hydroxy-1-cyclohexylproline isobutyl ester (A-2-03) and 4-hydroxy-1-diphenylmethylproline isobutyl ester hydrobromide (A-2-04) against the specifically tested cell lines is higher by many times over compared to that of the comparative substance CHP. In particular, cis-4-hydroxy-1,1-dimethylproline ethyl ester iodide (A-1-01) showed a specifically higher activity against the pancreas adenocarcinoma cell lines (BX PC3) than cis-4-hydroxy-1-proline.

The determination of the IC₅₀ (the concentration of active substance required to achieve a 50% inhibition) is a relevant parameter in the measurement of the pharmacological effectiveness of an active substance. The use of the substances cis-4-hydroxy-L-proline ethyl ester, cis-4-hydroxy-L-proline isobutyl ester, cis-4-hydroxy-1-diphenylmethylproline isobutyl ester hydrobromide and cis-4-hydroxy-1,1-dimethylproline ethyl ester iodide might have immense therapeutic advantages over other substances such as cis-4-hydroxy-L-proline and/or cis-4-hydroxy-1-methylproline.

In this event, the required therapeutic dosages would be much lower, and accordingly, pharmaceutical oral formulations used once a day (low dose once a day) rather than many times per day would be possible, for example. This is important to the patients' quality of life, cost of therapy and patient compliance.

Starting from the results of the determination of the IC₅₀ (the concentration of active substance required to achieve a 50% inhibition) it was possible to demonstrate that the use of the substances cis-4-hydroxy-L-proline ethyl ester, cis-4-hydroxy-L-proline isobutyl ester, cis-4-hydroxy-1-diphenylmethylproline isobutyl ester hydrobromide and cis-4-hydroxy-1,1-dimethylproline ethyl ester iodide has immense therapeutic advantages over other substances such as cis-4-hydroxy-L-proline and/or cis-4-hydroxy-1-methylproline.

Even more surprisingly, it was possible to demonstrate that the combination of cis-4-hydroxy-L-proline and cis-4-hydroxy-1-methyl-L-proline has an antagonistic effect on the specific cell lines Colo205, SW620 and T47D, the former being colon cancer cell lines and the last one being a breast cancer cell line. A proliferation test—as described above—with an initial dilution of 400 μg/ml was carried out, either alone or in combination with 400, 200 or 100 μg of CHP. The cell lines used are illustrated in Table 3. The column “Con” shows the % inhibition of proliferation (minus signs) by the varying concentrations of A0.21. In general, the results show that CHP changes the antiproliferative effect of A0.21 to the opposite. Frequently, the proliferation is increased, or the inhibition achieved with specific substances is lower when using combined agents. The effect of CHP alone is shown on the left in the table, below each specified cell line, for the highest concentration of CHP (400 μg/ml). The values in Table 3 show that A0.21 and CHP have an antagonistic effect under the specified conditions at the relevant and important concentrations.

TABLE 3
Combination of CHP with A0.21 (4-hydroxy-1-methylproline)

| | Con | CHP400 | CHP200 | CHP100 | (A021) |
|---|---|---|---|---|---|
| Colo205 | −8.2 | −9.7 | −8.0 | −4.8 | (400) |
| | −4.0 | −2.3 | +2.7 | +10.5 | (200) |
| (−46%) | −3.5 | −0.7 | +6.7 | +16.6 | (100) |
| T47D | −0.4 | +9.6 | +11.2 | −2.9 | (400) |
| | −1.2 | +35.6 | +31.3 | +25.1 | (200) |
| (−17.4%) | −1.8 | +25.2 | +37.5 | +23.6 | (100) |
| SW620 | −5.2 | −1.4 | +17.5 | −10.9 | (400) |
| | −6.3 | +13.9 | +34.1 | +3.8 | (200) |
| (−14%) | −5.8 | +12.2 | +17.3 | +18.6 | (100) |

Furthermore, (R)-(+)-α,α-diphenyl-2-pyrrolidinemethanol and (S)-(−)-α,α-diphenyl-2-pyrrolidinemethanol were tested. Table 4 shows the values of both enantiomers. The results are given as IC₅₀ (μg/ml).

TABLE 4
Diphenyl-2-pyrrolidinemethanol

| Cell lines | R enantiomer 231.01 | S enantiomer 231.02 |
|---|---|---|
| T47D breast cancer | 130 | 190 |
| Colo205 colon cancer | 45 | 45 |
| BxPC3 pancreas cancer | 60 | 50 |
| Pana -1 | 80 | 55 |
| pancreas cancer | | |
| MIAPaCa2 | 50 | 50 |
| pancreas cancer | | |

Furthermore, tests with the compounds of the invention were carried out, which tests are explained in the following with reference to cis-4-hydroxy-L-proline.

cis-4-Hydroxy-L-proline was repeatedly administered orally to rats over a period of 28 days. cis-4-Hydroxy-L-proline was analyzed in serum and urine samples using the LC/MS technique.

It was determined that the level of cis-4-hydroxy-L-proline in serum or urine rapidly dropped after the repeated administrations. The determination of the reduction of the cis-4-hydroxy-L-proline level by means of the LC/MS technique was associated with a detection of isomers and metabolites of cis-4-hydroxy-L-proline in the investigated samples.

Surprisingly, it was possible to detect the following biotransformations of cis-4-hydroxy-L-proline:

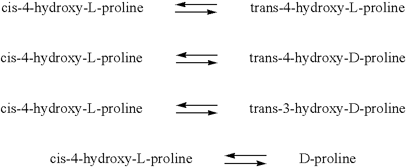

The above biotransformation is catalyzed by hitherto unknown CHP isomerases and/or CHP epimerases.

The formation of trans-4-hydroxy-L-proline or other products of the biotransformation is disadvantageous because they frequently lack pharmacological activity.

Specific inhibitors of CHP isomerases and/or CHP epimerases can prevent the biotransformation or conversion of cis-4-hydroxy-L-proline into trans-4-hydroxy-L-proline, trans-4-hydroxy-D-proline, trans-3-hydroxy-D-proline or, generally, into D-proline, thereby maintaining the concentration of cis-4-hydroxy-L-proline in the organism on a high level.

When additionally administering CHP isomerases and/or CHP epimerases in association with oral or other administration of cis-4-hydroxy-L-proline simultaneously or in a time-shifted manner, the dosage of cis-4-hydroxy-L-proline or derivatives thereof can be lower, because loss as a result of biotransformation, i.e. isomerization and epimerization, in the organism is avoided.

1. A compound of general formula (I), 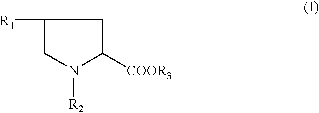
wherein
R₁ is a hydroxy, aryl or amino acid group,
R₂ is hydrogen, an alkyl (C₁-C₄), a substituted alkyl (C₁-C₄) group, a dialkyl (C₁-C₄), a cyclohexyl, a phenyl or diphenyl group,
R₃ is an alkyl (C₂-C₅) group, and/or salts thereof,
with the proviso that, if R₁ is a hydroxy group, R₂ is not a methyl group, said compound being selected from the group comprising 4-hydroxy-1,1-dimethylproline ethyl ester iodide, 4-hydroxyproline isobutyl ester, 4-hydroxy-1,1-dimethylproline isobutyl ester iodide, 4-hydroxy-1-cyclohexylproline isobutyl ester, 4-hydroxyl-1-diphenylmethylproline isobutyl ester hydrobromide, 4-hydroxy-1-methylproline ethyl ester, 4-hydroxy-1-methylproline isobutyl ester and/or 1-methyl-4-phenylaminocarbonyloxyproline isobutyl ester, and, if R₁ is a hydroxy group, said compounds may have a methyl group in position R₂.

2. A pharmaceutical agent comprising a compound according to claim 1, optionally together with conventional auxiliaries, preferably pharmaceutically acceptable carriers, adjuvants and/or vehicles.

3. The pharmaceutical agent according to claim 2, wherein
the carriers are selected from the group comprising fillers, diluents, binders, humectants, disintegrants, dissolution retarders, absorption enhancers, wetting agents, adsorbents and/or lubricants.

4. The pharmaceutical agent according to claim 2, wherein
the carriers are liposomes, siosomes and/or niosomes.

5. The pharmaceutical agent according to claim 2, wherein
the agent additionally comprises a chemotherapeutic agent.

6. The pharmaceutical agent according to claim 5, wherein
the chemotherapeutic agent is selected from the group comprising oxoplatin, cisoxoplatin, taxol, gemcitabine, vinorelbine, paclitaxel, cyclosporin and/or a combination thereof.

7. The pharmaceutical agent according to claim 2, further comprising one or more additional agents from the group of antiviral, antimycotic, antibacterial and/or immunostimulatory agents.

8. (canceled)

9. Method for the diagnosis, prophylaxis, follow-up, therapy, and/or aftercare of a disease associated with cell growth, cell differentiation and/or cell division, comprising
administering to a person in need thereof and/or benefiting therefrom 4-hydroxyproline ethyl ester, 4-hydroxy-1,1-dimethylproline ethyl ester iodide, 4-hydroxyproline isobutyl ester, 4-hydroxy-1,1-dimethylproline isobutyl ester iodide, 4-hydroxy-1-cyclohexylproline isobutyl ester, 4-hydroxy-1-diphenylmethylproline isobutyl ester hydrobromide, 4-hydroxy-1-methylproline, 4-hydroxy-1-methylproline ethyl ester, 4-hydroxy-1-methylproline isobutyl ester, 1-methyl-4-phenylaminocarbonyloxyproline, 1-methyl-4-phenylaminocarbonyloxyproline isobutyl ester, (R)-(+)-α,α-diphenyl-2-pyrrolidinemethanol and/or (S)-(−)-α,α-diphenyl-2-pyrrolidinemethanol and/or derivatives, metabolites, enantiomers and/or isomers thereof in a diagnosis, prophylaxis, follow-up, therapy, and/or aftercare of diseases associated with cell growth, cell differentiation and/or cell division effective amount, wherein said disease is a tumor.

10. The method of claim 9, wherein
the tumor disease is a neoplastic tumor, an inflammatory tumor, an abscess, effusion and/or edema.

11. The method of claim 9, wherein
the tumor is a solid tumor or a leukemia.

12. The method of claim 11, wherein
the solid tumor is a tumor of the urogenital tract and/or gastrointestinal tract.

13. The method of claim 9, wherein
the tumor is a colon carcinoma, stomach carcinoma, pancreas carcinoma, small intestine carcinoma, ovarian carcinoma, cervical carcinoma, lung carcinoma, prostate carcinoma, mammary carcinoma, renal cell carcinoma, a brain tumor, head-throat tumor, liver carcinoma, and/or a metastase of the above tumors.

14. The use according to method of claim 11, wherein
the solid tumor is a mammary, bronchial, colorectal, and/or prostate carcinoma and/or a metastase of the above tumors.

15. The method of claim 12, wherein
the tumor of the urogenital tract is a bladder carcinoma and/or a metastase of such tumors.

16. The method of claim 9, wherein
said follow-up is monitoring the effectiveness of an anti-tumor treatment.

17. A method for the prophylaxis, prevention, diagnosis, attenuation, therapy, follow-up and/or aftercare of metastasizing, invasion, infiltration, tumor growth and/or angiogenesis comprising
administering to a person in need thereof and/or benefiting therefrom at least one compound according to claim 1 and/or a pharmaceutical agent according to claim 2 in a prophylaxis, prevention, diagnosis, attenuation, therapy, follow-up and/or aftercare of metastasizing, invasion, infiltration, tumor growth and/or angiogenesis effective amount.

18. The method of claim 17, wherein said follow-up is monitoring the effectiveness of an anti-tumor treatment.

19. The method of claim 17, wherein the methods are used as part of a combined therapy.

20. The combined therapy of claim 19 further comprising a chemotherapy, a treatment with cytostatic agents and/or a radiotherapy.

21. The the combined therapy of claim 19 further comprising an adjuvant, biologically specified form of therapy.

22. The method of claim 21, wherein
said form of therapy is an immune therapy.

23. The method of claim 17, wherein said method increases sensitivity of tumor cells to cytostatic agents and/or radiation.

24. The method of claim 17, wherein said method inhibits viability, proliferation rate of cells in order to induce apoptosis and/or cell cycle arrest.

25. The pharmaceutical agent of claim 2, wherein said agent is in form of a gel, poudrage, powder, tablet, sustained-release tablet, premix, emulsion, brew-up formulation, drops, concentrate, granulate, syrup, pellet, bolus, capsule, aerosol, spray and/or inhalant and/or inhalant and applied in this form.

26. The pharmaceutical agent of claim 2, wherein said agent is present in a preparation at a concentration of from 0.1 to 99.5, preferably from 0.5 to 95.0, and more preferably from 20.0 to 80.0 weight percent.

27. The pharmaceutical agent of claim 26, wherein
the preparation is employed orally, subcutaneously, intravenously, intramuscularly, intraperitoneally and/or topically.

28. The method of claim 17, wherein the pharmaceutical agent is employed in overall amounts of more than 0.1 mg per kg body weight per 24 hours.

29. The method of claim 28, wherein the pharmaceutical agent is employed in overall amounts of 0.05 to 500 mg per kg, preferably 5 to 100 mg per kg body weight per 24 hours.

30. A method for the treatment of a tumor disease, comprising contacting
an organism is contacted with an effective amount of a compound according to claim 1.

31. A method for the inhibiting collagen IV and/or glutathione S transferase (GST) comprising administering to a cell or person benefiting from such inhibition a compound according to claim 1 in an collagen IV and/or glutathione S transferase (GST) inhibiting amount.

32. A method for the preparation of a compound according to claim 1, wherein
1-methyl-4-phenylaminocarbonyloxyproline ethyl ester is obtained by reacting 4-hydroxy-1-methylproline ethyl ester and phenyl isocyanate in acetonitrile.

33. A method for the preparation of a compound according to claim 1, wherein
1-methyl-4-phenylaminocarbonyloxyproline isobutyl ester is obtained by reacting 4-hydroxy-1-methylproline isobutyl ester and phenyl isocyanate in acetonitrile.

34. A method for the preparation of a compound according to claim 1, wherein
4-hydroxy-1-methylproline is obtained by reacting 4-hydroxyproline in formalin with Pd/C in a hydrogenation apparatus.

35. A method for the preparation of a compound according to claim 1, wherein
4-hydroxy-1-methylproline ethyl ester is obtained by reacting 4-hydroxyproline ethyl ester and formalin in ethanol.

36. A method for the preparation of a compound according to claim 1, wherein
4-hydroxy-1-methylproline isobutyl ester is obtained by reacting formalin, Pd/C and ethanol and 4-hydroxyproline isobutyl ester.

37. A method for the preparation of a compound according to claim 1, wherein
4-hydroxy-1-methylproline isobutyl ester is obtained by reacting formalin and 4-hydroxyproline isobutyl ester in the presence of Pd/C in ethanol.

38. A method for the preparation of a compound according to claim 1, wherein
cis-4-hydroxy-L-proline ethyl ester is obtained by contacting 4-hydroxyproline with HCl in ethanol.

39. A method for the preparation of a compound according to claim 1, wherein
cis-4-hydroxy-L-proline isobutyl ester is obtained by reacting 4-hydroxyproline in isobutanol.

40. A method for the preparation of a compound according to claim 1, wherein
4-hydroxy-1,1-dimethylproline ethyl ester iodide is obtained by reacting hydroxyproline ethyl ester in acetonitrile, methyl iodide and triethylamine.

41. A method for the preparation of a compound according to claim 1, wherein
4-hydroxy-1,1-dimethylproline isobutyl ester iodide is obtained by reacting 4-hydroxyproline isobutyl ester and methyl iodide in triethylamine and acetonitrile.

42. A method for the preparation of a compound according to claim 1, wherein
4-hydroxy-1-alkylproline ester bromide is obtained by suspending 4-hydroxyproline ester in acetonitrile and contacting with the corresponding alkyl bromide in the presence of ether.

43. A method for the preparation of a compound according to claim 1, wherein
4-hydroxy-1-cyclohexylproline isobutyl ester is obtained by dissolving the corresponding hydrobromide in chloroform and contacting with gaseous ammonia.

44. A method for the preparation of a compound according to claim 1, wherein
4-hydroxy-1-diphenylmethylproline isobutyl ester hydrobromide is obtained by contacting 4-hydroxyproline isobutyl ester, methyl iodide, triethylamine in acetonitrile.

45. A kit comprising at least one compound according to claim 1, optionally together with information for combining the contents of the kit.

46. (canceled)
